(19) United States
(12) Patent Application Publication
(10) Pub. No.: US 2010/0151553 A1
(43) Pub. Date: Jun. 17, 2010

(54) METHOD OF DETECTION OF BIOANALYTES BY ACOUSTO-MECHANICAL DETECTION SYSTEMS COMPRISING THE ADDITION OF LIPOSOMES

(76) Inventors: Jason W. Bjork, Newport, MN (US); Samuel J. Gason, Victoria (AU); Michael C. Palazzotto, Woodbury, MN (US); Stephen B. Roscoe, Woodbury, MN (US)

Correspondence Address: 3M INNOVATIVE PROPERTIES COMPANY, PO BOX 33427, ST. PAUL, MN, 55133-3427, US

(21) Appl. No.: 12/521,611

(22) PCT Filed: Dec. 28, 2007
(86) PCT No.: PCT/US07/89053

§ 371 Date: Feb. 23, 2010

Related U.S. Application Data
(60) Provisional application No. 60/882,899, filed on Dec. 29, 2006.

(51) Int. Cl. C12N 13/00

(52) U.S. Cl. 435/173.7

(57) ABSTRACT

Methods for detecting target biological analytes within sample material using acousto-mechanical energy generated by a sensor are disclosed. The acousto-mechanical energy may be provided using an acousto-mechanical sensor, e.g., a surface acoustic wave sensor such as, e.g., a shear horizontal surface acoustic wave sensor (e.g., a LSH-SAW sensor). The detection of the target biological analytes in sample material are enhanced by contacting the target biological analyte and/or the sensor surface with liposomes that amplify the sensor sensitivity by (1) modifying the rheological properties of the fluid near the sensor surface; (2) changing the mass attached to the surface; and/or (3) modifying the dielectric properties of the fluid near the sensor surface, the sensor surface itself and/or any intervening layers on the sensor surface.

### GOVERNMENT RIGHTS

The U.S. Government may have certain rights to this invention under the terms of DAAD 13-03-C-0047 granted by Department of Defense.

### BACKGROUND

In the case of acousto-mechanical sensors, many biological analytes are introduced to the sensors in combination with a liquid carrier. The liquid carrier may undesirably reduce the sensitivity of the acousto-mechanical detection systems. Furthermore, the selectivity of such sensors may rely on properties that cannot be quickly detected, e.g., the test sample may need to be incubated or otherwise developed over time.

To address that problem, selectivity can be obtained by binding a target biological analyte to, e.g., a detection surface. Selective binding of known target biological analytes to detection surfaces can, however, raise issues when the sensor used relies on acousto-mechanical energy to detect the target biological analyte.

Acoustic wave sensors are so named because their detection mechanism is a mechanical, or acoustic, wave. As the acoustic wave propagates through or on the surface of the material, any changes to the characteristics of the propagation path affect the velocity and/or amplitude of the wave. Changes in velocity can be monitored by measuring the frequency or phase characteristics of the sensor and can then be correlated to the corresponding physical quantity being measured.

Acoustic wave devices are described by the mode of wave propagation through or on a piezoelectric substrate. When the acoustic wave propagates on the surface of the substrate, it is known as a surface wave. The surface acoustic wave sensor (SAW) and the shear-horizontal surface acoustic wave (SH-SAW) sensor are the most widely used surface wave devices. One of the important features of a SH-SAW sensor is that it allows for sensing in liquids.

Shear horizontal surface acoustic wave sensors are designed to propagate a wave of acousto-mechanical energy along the plane of the sensor detection surface. In some systems, a waveguide may be provided at the detection surface to localize the acousto-mechanical wave at the surface and increases the sensitivity of the sensor (as compared to a non-waveguided sensor). This modified shear horizontal surface acoustic wave is often referred to as a Love-wave shear horizontal surface acoustic wave sensor (“LSH-SAW sensor”).

Such sensors have been used in connection with the detection of chemicals and other materials where the size of the target analytes is relatively small. As a result, the mass of the target analytes is largely located within the effective wave field of the sensors (e.g., about 60 nanometers (nm) for a sensor operating at, e.g., a frequency of 103 Megahertz (MHz) in water).

For these sensors, the adsorption of an analyte on the surface perturbs the acoustic waves propagated across the sensor, allowing the detection of an analyte. These perturbations can be measured as changes in the phase and attenuation of the device. In a typical sensing experiment, the sensor is stabilized for some time, the analyte of interest is injected over the sensor and the change in phase and attenuation is measured. The change in phase and/or the change in attenuation is expected to correlate to the presence and possibly the concentration of the target analyte.

The sensors can experience limitations in detection, particularly at lower concentrations of the target analyte in a sample. Several reasons exist for this effect including the fraction of the analyte present that is actually captured on the sensor surface; the mass and/or size of the captured target analyte; and the inherent sensitivity of the SAW device. Thus, a need still exists for improvements in the detection of target analytes using acousto-mechanical detection systems.

### SUMMARY OF THE INVENTION

The present invention provides methods for enhancing the detection of target biological analytes within sample material using acousto-mechanical energy generated by a sensor. The method includes attaching a liposome to the target biological analyte and/or detection surface of an acousto-mechanical device to amplify the signal response from the acousto-mechanical sensor.

The acousto-mechanical energy may preferably be provided using an acousto-mechanical sensor, e.g., a surface acoustic wave sensor such as a shear horizontal surface acoustic wave sensor (e.g., a LSH-SAW sensor), although other acousto-mechanical sensor technologies may be used in connection with methods of the present invention. Improvements in the detection limit may be increased using the methods described herein.

A method of detecting a target biological analyte is provided, the method comprising providing a system comprising an acousto-mechanical device comprising a detection surface with a capture agent located on the detection surface, wherein the capture agent is capable of selectively attaching the target biological analyte to the detection surface; contacting the detection surface of the acousto-mechanical device with a sample material that may contain the target biological analyte; selectively attaching the target biological analyte to the detection surface; contacting the target biological analyte and/or detection surface with a liposome; and operating the acousto-mechanical device to detect the attached target biological analyte while the detection surface is submersed in liquid.

As used herein and in the appended claims, the singular forms “a,” “and,” and “the” include plural referents unless the context clearly dictates otherwise. Thus, for example, reference to “a target biological analyte” includes a plurality of target biological analytes and reference to “the detection chamber” includes reference to one or more detection chambers and equivalents thereof known to those skilled in the art.

The terms “comprises” and variations thereof do not have a limiting meaning where these terms appear in the description or the claims.

These and other features and advantages of the detection systems and methods of the present invention may be described in connection with various illustrative embodiments of the invention below.

### BRIEF DESCRIPTION OF THE DRAWINGS

FIG. 1 is a representation of an acoustic sensor.

FIG. 2 is a schematic diagram of one exemplary detection apparatus including a biosensor.

FIG. 3 is a schematic diagram of a detection apparatus including a biosensor.

FIG. 4 is a schematic diagram of an acoustic sensor detection system.

FIG. 5 is a graph of changes in a QCM sensor with the addition of the reagents as described in Example 8.

FIG. 6 is a graph of changes in dissipation of a QCM sensor with the addition of the reagents as described in Example 8.

### DETAILED DESCRIPTION OF EXEMPLARY EMBODIMENTS OF THE INVENTION

In the following detailed description of exemplary embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention.

The methods described herein use liposomes for the amplification of the response from an acousto-mechanical wave device. The method includes attaching a liposome to the target biological analyte and/or detection surface of an acousto-mechanical device to amplify the signal response from the acousto-mechanical sensor. The method may include attaching the target biological analyte (if present) to the liposome, and subsequently contacting the sensor surface with the target biological analyte/liposome conjugate. Alternatively, the method may include contacting the sensor surface with the target biological analyte and subsequently contacting the sensor surface (with the attached target biological analyte) with the liposomes. In some embodiments, the method may include the step of contacting the sensor surface (with both target biological analyte and liposomes attached) with a rupture agent.

The use of liposomes to bind to the target analyte and/or the sensor surface provides an enhancement in detection of target biological analytes on the sensor surface. The sensor response is significantly increased when exposed to the liposome, thereby increasing the sensitivity of the acousto-mechanical wave device. A target biological analyte is bound to the sensor surface and produces a characteristic sensor response dependent on the mass deposited and visco-elastic property changes at the sensor-liquid interface. The size of the response per unit mass is then used to define the device sensitivity. Adding a liposome to the target biological analyte bound to the sensor surface may produce a significant property change that may increase the device sensitivity. The liposome amplifies the signal by (1) modifying the rheological properties of the fluid near the sensor surface; (2) changing the mass attached to the surface; and/or (3) modifying the dielectric properties of the fluid near the sensor surface, the sensor surface itself and/or any intervening layers on the sensor surface.

Liposomes are generally two-phase materials that encapsulate a material of a different property than the liposome and/or the bulk phase, and function to separate the material with the different property from the bulk phase. The liposomes may be ruptured to release the captured material into solution, which may in some circumstances create a significant change in the bulk solution property (such as the density, viscosity, dielectric constant, pH, etc.). In some embodiments, the liposomes may rupture upon attachment to the sensor surface, producing an amplified signal that correlates to the concentration of the target biological analyte. In other embodiments, the liposomes remain intact upon attachment to the target biological analyte and/or the sensor detection surface to produce the amplification in signal.

The liposome may encapsulate a material that changes the viscoelastic properties of the medium such as a viscous polymer, the dielectric constant of the medium, such as salt(s) or ionic polymers, and/or adds mass to the sensor surface such as gold nanoparticles or magnetic particles. In some embodiments, the material encapsulated in the liposome is a gelling agent.

Numerous rupture agents and methods can be employed to rupture liposomes, or otherwise release the contents. The liposomes can be ruptured by physical methods, such as heat or rapid freezing or ultrasonic radiation. Appropriately designed liposomes can be made sensitive to electromagnetic radiation at various wavelengths (see, for example: Collier et. al. J. Amer. Chem. Soc., 2001, 123, 9463.

A rupture agent may also be used to lyse the liposome upon attachment to the sensor surface, either directly or as attached to the target biological analyte. Liposomal rupture agents can be any natural or synthetic agent that ruptures or lyses a liposome, or generates transient or long-lasting pores in the bilayer membrane of a liposome, or otherwise disrupts the membrane in such a way that the contents of the liposome are released, or molecules outside the liposome become internalized. The rupture agent may simply change the salt concentration or pH, or may function as surfactants such as TRITON-X 100 (trademarked), or n-octyl-B-D-glucopyranoside. Various natural agents such as enzymes, are known to lyse liposomes, as are natural and synthetic cytolytic peptides such as melittin, alamethicin, magainin, and GALA or natural proteins such as streptolysin or lysteriolysin.

The rupture agent may be the same as the fractionating agents mentioned herein. In one embodiment, the rupture agent is TRITON-X 100 (octyl phenol ethoxylate), commercially available from Rohm & Haas Co.

As discussed above, the use of acousto-mechanical energy to detect the presence or absence of target biological analyte in sample material is affected by the ability to effectively couple the target biological analyte to the detection surface such that the acousto-mechanical energy from the sensor is affected in a detectable manner. As used herein, “target biological analyte” may include, e.g., microorganisms (e.g., bacteria, viruses, endospores, fungi, protozoans, etc.), proteins, peptides, amino acids, fatty acids, nucleic acids, carbohydrates, hormones, steroids, lipids, vitamins, etc.

The detection methods of the present invention may, in some embodiments, provide a variety of techniques for detecting the target biological analytes in sample material. In one embodiment, the method includes optionally fractionating or disassembling the target biological analytes in the sample material (e.g., lysing the target biological analyte), contacting the target analyte with the surface of an acousto-mechanical sensor, and contacting the liposome with the analyte on the surface of the acousto-mechanical sensor. In another embodiment, the method includes optionally fractionating or disassembling the target biological analytes in the sample material (e.g., lysing the target biological analyte), contacting the target biological analyte with the liposome to form a target analyte/liposome conjugate, and contacting the target analyte/liposome conjugate with the analyte on the surface of the acousto-mechanical sensor. The acousto-mechanical sensor is coated with a capture agent with an affinity to the target analyte and/or the liposome.

The target biological analyte may be obtained from sample material that is or that includes a test specimen obtained by any suitable method and may largely be dependent on the type of target biological analyte to be detected. For example, the test specimen may be obtained from a subject (human, animal, etc.) or other source by e.g., collecting a biological tissue and/or fluid sample (e.g., blood, urine, feces, saliva, semen, bile, ocular lens fluid, synovial fluid, cerebral spinal fluid, pus, sweat, exudate, mucous, lactation milk, skin, hair, nails, etc.). In other instances, the test specimen may be obtained as an environmental sample, product sample, food sample, etc. The test specimen as obtained may be a liquid, gas, solid or combination thereof. Before delivery to the systems and methods of the present invention, the sample material and/or test specimen may be subjected to prior treatment, e.g., dilution of viscous fluids, concentration, filtration, distillation, dialysis, addition of reagents, chemical treatment, etc.

The capture of the target biological analyte to the surface of the sensor and/or the liposome is accomplished by using a capture agent with an affinity to the target biological analyte. The capture agent may bind to the target analyte by specific or non-specific binding. For instance, streptavidin may be used to capture Protein A-biotin. Similarly, other target analytes can be captured by attaching and/or coating biotinylated proteins such as a streptavidin-coated liposomes with a biotinylated antibody that is specific to the target biological analyte.

The data generated in experiments with SAW sensors is typically gathered in the frequency domain. The data can be transformed into the time domain and a time gating algorithm can be performed. In a typical algorithm, the gates are applied to filter out undesirable time signals, and the data can then be transformed back into the frequency domain. For the case in which the sensor has a reference channel, the reference channel signal can be subtracted from the active channel signal to filter out undesirable noise.

The target biological analyte is attached to the sensor surface and/or the liposome via a capture agent with selective affinity to the target biological analyte. The target biological analyte may be attached in combination with fractionating/disassembly techniques (where, e.g., the particles could attach to fragments of a cell wall, etc.). In some embodiments, the target biological analyte is fractionated or otherwise disassembled into smaller fragments or particles such that an increased percentage of the target biological analyte bound to the sensor surface can be retained within the effective wave field of the acousto-mechanical sensor and/or effectively coupled with the detection surface of the acousto-mechanical sensor.

The fractionating or disassembly may be accomplished chemically, mechanically, electrically, thermally, or through combinations of two or more such techniques. Examples of some potentially suitable chemical fractionating techniques may involve, e.g., the use of one or more enzymes, hypertonic solutions, hypotonic solutions, detergents, etc. Examples of some potentially suitable mechanical fractionating techniques may include, e.g., exposure to sonic energy, mechanical agitation (e.g., in the presence of beads or other particles to enhance breakdown), etc. Thermal fractionating may be performed by, e.g., heating the target biological agent. Other fractionating/disassembly techniques may also be used in connection with the present invention. U.S. patent application Ser. No. 11/015,166, titled “Method of Enhancing Signal Detection of Cell-Wall Components of Cells”, filed on Dec. 17, 2004, describes the use of lysing as one method of fractionating a target biological analyte that may be used in connection with the present invention.

### Liposomes

Liposomes, also known as vesicles, are designed to encapsulate a material of a different property and separate the material with the different property from the bulk phase. They are typically spherical in shape, and preferably have an average particle size (i.e., the average of the longest dimension, which is the diameter for spherical particles) of no greater than 5000 nanometers (nm).

Suitable types of liposomes may be prepared from, for example, phospholipids such as phosphatidylserine, phosphatidylinositol, phosphatidylethanolamine, phosphatidylcholines, phosphatidylglycerol, phosphatidic acid, phosphatidylmethanol, cardiolipin, ceramide, cholesterol, cerebroside, lysophosphatidylcholine, D-erythrosphingosine, sphingomyelin, dodecyl phosphocholine, N-biotinyl phosphatidylethanolamine, synthetic analogs of these molecules, derivatives of these molecules, and combinations thereof. In a preferred embodiment, the liposomes comprise 1,2-dipalmitoyl-sn-glycero-3-phosphocholine (DPPC); 1,2-dipalmitoyl-sn-glycero-3-phosphoethanolamine-N-(Cap-biotinyl) (16:0 Biotinyl-Cap-PE); and combinations thereof.

Liposomes may be prepared according to any of the well known conventional processes. For example, liposomes may be made by depositing a thin film of lipid on the inner wall of a flask, adding an aqueous phase, and shaking vigorously by hand. Another method may include, for example, sonication of a lipid film in an aqueous solution, followed by extrusion through a series of filters of decreasing pore size. Yet another method of making liposomes is to dialyze an aqueous solution of lipids in the presence of a detergent such as sodium cholate. As the detergent is depleted, the lipids form liposomes. Still another method is based on high pressure homogenization of a lipid solution using commercially available equipment. Additional methods may include, for example, re-hydration of freeze-dried vesicles and reverse-phase evaporation. Descriptions and protocols for these methods are well known to those of skill in the art. See, for example, Liposomes: A Practical Approach (2^{nd} edition, 2003), edited by Vladimir Torchilin and Volkmar Weissig, Oxford University Press, Oxford, UK.

Other components may be added in conjunction with the liposomes for improving sensor sensitivity. For example, the methods described herein may also include the use of magnetic particles to increase sensor sensitivity as described in U.S. Ser. No. 60/882,816, filed on Dec. 29, 2006, entitled “, entitled “Methods of Detection Using Acousto-Mechanical Detection Systems.” Use of a magnetic field generator with magnetic particles bound to the liposomes and/or target biological analytes may work to increase capture efficiency of the target biological analyte and/or liposome on the sensor surface using magnetophoresis. Because the target analytes and/or liposomes are bound on the magnetic particles drawn to the sensor surface, the target analytes are moved to the surface at much higher rates than other constituents of the sample.

Other reagents may also be added that cause a change in the viscous, elastic, and/or viscoelastic properties of the sample material in contact with the detection surface. Examples of some suitable mass-modification techniques may be, e.g., the use of fibrinogen in combination with Staphylococcus species as described in, e.g., U.S. Patent Application Ser. No. 60/533,171, filed on Dec. 30, 2003 and U.S. patent application Ser. No. 10/960,491, filed on Oct. 7, 2004.

### Selective Attachment

The detection systems and methods of the present invention may preferably provide for the selective attachment of target biological analyte to the detection surface or to another component, such as the liposome, that can be coupled to the detection surface. For example, the liposome wall may be functionalized with a specific binding agent, such as an antibody, to facilitate binding the liposome to the target biological analyte.

Selective attachment may be achieved by a variety of techniques. Some examples may include, e.g., antigen-antibody binding; affinity binding (e.g., avidin-biotin, nickel chelates, glutathione-GST); covalent attachment (e.g., azlactone, trichlorotriazine, phosphonitrilic chloride trimer or N-sulfonylaminocarbonyl-amide chemistries); etc.

The selective attachment of a target biological analyte may be achieved directly, i.e., the target biological analyte itself is selectively attached to the detection surface. Examples of some such direct selective attachment techniques may include the use of capture agents, e.g., antigen-antibody binding (where the target biological analyte itself includes the antigen bound to an antibody immobilized on the detection surface), DNA capture, etc.

The selective attachment may also be indirect, i.e., where the target biological analyte is selectively attached to the liposome, with the resulting analyte/liposome conjugate then selectively attached or attracted to the detection surface. The indirect selective attachment technique includes selectively binding liposomes to the target biological analyte, and having then the analyte/liposome conjugate retained on the detection surface.

In connection with selective attachment, it may be preferred that systems and methods of the present invention provide for low non-specific binding of other biological analytes or materials to, e.g., the detection surface. Non-specific binding can adversely affect the accuracy of results obtained using the detection systems and methods of the present invention. Non-specific binding can be addressed in many different manners. For example, biological analytes and materials that are known to potentially raise non-specific binding issues may be removed from the test sample before it is introduced to the detection surface. In another approach, blocking techniques may be used to reduce non-specific binding on the detection surface.

Because selective attachment may often rely on coatings, layers, etc. located on the acousto-mechanical detection surface, care must be taken that these materials are relatively thin and do not dampen the acousto-mechanical energy to such a degree that effective detection is prevented.

Another issue associated with selective attachment is the use of what are commonly referred to as “immobilization” technologies that may be used to hold or immobilize a capture agent on the surface of, e.g., the acousto-mechanical sensor itself. The detection systems and methods of the present invention may involve the use of a variety of immobilization technologies.

As discussed herein, the general approach is to coat or otherwise provide the detection surface of an acousto-mechanical sensor device with capture agents such as, e.g., antibodies, peptides, aptamers, or any other capture agent that has affinity for the target biological analyte and/or the liposome. The surface coverage and packing of the capture agent on the surface may determine the sensitivity of the sensor. The immobilization chemistry that links the capture agent to the detection surface of the sensor may play a role in the packing of the capture agents, preserving the activity of the capture agent (if it is a biomolecule), and may also contribute to the reproducibility and shelf-life of the sensor.

If the capture agents are proteins or antibodies, they can nonspecifically adsorb to a surface and lose their ability (activity) to capture the target biological analyte and/or liposome. A variety of immobilization methods may be used in connection with acousto-mechanical sensors to achieve the goals of high yield, activity, shelf-life and stability. These capture agents may preferably be coated using glutaraldehyde cross-linking chemistries, entrapment in acrylamide, or general coupling chemistries like carbodiimide, epoxides, cyano bromides etc. Depending on the capture agent used, the concentration of capture agent sensor surface may become important in optimizing the sensor response.

Apart from the chemistry that binds to the capture agent and still keeps it active, there are other surface characteristics of any immobilization chemistries used in connection with the present invention that may need to be considered and that may become relevant in an acousto-mechanical sensor application. For example, the immobilization chemistries may preferably cause limited damping of the acousto-mechanical energy such that the immobilization chemistry does not prevent the sensor from yielding usable data. The immobilization chemistry may also determine how the antibody or protein is bound to the surface and, hence, the orientation of the active site of capture. The immobilization chemistry may preferably provide reproducible characteristics to obtain reproducible data and sensitivity from the acousto-mechanical sensors of the present invention.

Some immobilization technologies that may be used in connection with the systems and methods of the present invention may be described in, e.g., U.S. patent application Ser. Nos. 10/713,174, filed Nov. 14, 2003; 10/987,522, filed on Nov. 12, 2004; 60/533,162, filed on Dec. 30, 2003; 60/533,178, filed on Dec. 30, 2003, Ser. No. 10/896,392, filed on Jul. 22, 2004; Ser. No. 10/714,053, filed on Nov. 14, 2003; Ser. No. 10/987,075, filed on Nov. 12, 2004; Ser. No. 11/015,399 titled “Soluble Polymers as Amine Capture Agents and Methods”, filed on Dec. 17, 2004; Ser. No. 11/015,543 titled “Multifunctional Amine Capture Agents”, filed on Dec. 17, 2004; and PCT Publication No. WO2005/066092 titled “Acoustic Sensors and Methods”, filed on Dec. 17, 2004.

Immobilization approaches may include a tie layer between the waveguide on an acousto-mechanical substrate and the immobilization layer. One exemplary tie layer may be, e.g., a layer of diamond-like glass, such as described in International Publication No. WO 01/66820 A1 (David et al.).

### Acousto-Mechanical Sensors

The systems and methods of the present invention preferably detect the presence of target biological analyte in a test sample through the use of acousto-mechanical energy that is measured or otherwise sensed to determine wave attenuation, phase changes, frequency changes, and/or resonant frequency changes.

The acousto-mechanical energy may be generated using, e.g., piezoelectric-based surface acoustic wave (SAW) devices. As described in, e.g., U.S. Pat. No. 5,814,525 (Renschler et al.), the class of piezoelectric-based acoustic mechanical devices can be further subdivided into surface acoustic wave (SAW), acoustic plate mode (APM), or quartz crystal microbalance (QCM) devices depending on their mode of detection.

The methods described herein employ an acoustic sensor, and more specifically, an acoustic mechanical biosensor, that detects a change in at least one physical property and produces a signal in response to the detectable change. In some embodiments, the acoustic mechanical biosensor employed herein is a surface acoustic wave (SAW) biosensor. In these devices an acoustic wave is generated from an interdigitated transducer (IDT) on a piezoelectric substrate either as a surface acoustic wave or as a bulk acoustic wave. A second IDT may convert the acoustic wave back to an electric signal for measurement. This is referred to as a delay line. Alternatively the device may operate as a resonator. The space between the two IDTs can be modified with a coating that may include reactive molecules for chemical or biosensing applications.

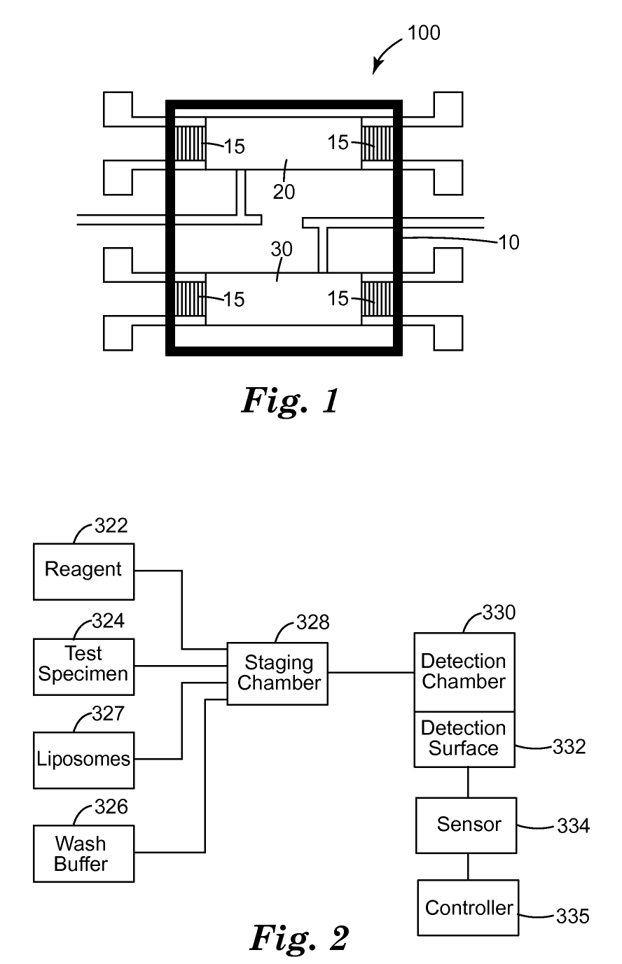

With reference to FIG. 1, in some embodiments the acoustic mechanical biosensor surface 100 between the IDTs 15 preferably comprises two delay lines. A first channel, i.e. the “active” channel 20 is provided for receipt of the test sample. The second channel, i.e. the “reference” channel 30 is provided as the baseline or control. Accordingly, the change in physical property is the difference between the active channel and the reference channel. When necessary, an acoustic waveguide 10 (only the boundaries of which are depicted in FIG. 1) typically covers the area between the IDTs as well as the IDTs themselves. The data may be transformed with mathematical algorithms in order to improve the sensitivity. Alternative configurations of an exemplary acoustic mechanical sensor include those disclosed in PCT Publication No. WO2005/075973 titled “Acousto-mechanical Detection Systems and Methods of Use”, filed Dec. 17, 2004.

Piezoelectric-based SAW biosensors typically operate on the basis of their ability to detect minute changes in mass or viscosity. As described in U.S. Pat. No. 5,814,525, the class of piezoelectric-based acoustic mechanical biosensors can be further subdivided into surface acoustic wave (SAW), acoustic plate mode (APM), or quartz crystal microbalance (QCM) devices depending on their mode of detection of mass changes.

In some embodiments, the acoustic mechanical biosensor includes a secondary capture agent or reactant (e.g., antibody) that attaches the target analyte to the surface of the piezoelectric acoustic mechanical biosensor. The propagation velocity of the surface wave is a sensitive probe capable of detecting changes such as mass, elasticity, viscoelasticity, conductivity and dielectric constant. Thus, changes in any of these properties results in a detectable change in the surface acoustic wave. That is, when a substance comes in contacts with, absorbs, or is otherwise caused to adhere to the surface coating of a SAW device, a corresponding response is produced.

APM can also be operated with the device in contact with a liquid. Similarly, an alternating voltage applied to the two opposite electrodes on a QCM (typically AT-cut quartz) device induces a thickness shear wave mode whose resonance frequency changes in proportion to mass changes in a coating material.

The direction of the acoustic wave propagation (e.g., in the plane parallel to the waveguide or perpendicular to the plane of the waveguide) is determined by the crystal-cut of the piezoelectric material from which the acoustic mechanical biosensor is constructed. SAW biosensors that have the majority of the acoustic wave propagating in and out of the plane (i.e., Rayleigh wave, most Lamb-waves) are typically not employed in liquid sensing applications since there is too much acoustic damping from the liquid contact with the surface.

For liquid sample mediums, a shear horizontal surface acoustic wave biosensor (SH-SAW) is preferably constructed from a piezoelectric material with a crystal-cut and orientation that allows the wave propagation to be rotated to a shear horizontal mode, i.e., in plane of the biosensor waveguide), resulting in reduced acoustic damping loss to the liquid in contact with the biosensor surface. Shear horizontal acoustic waves include, e.g., acoustic plate modes (APM), surface skimming bulk waves (SSBW), Love-waves, leaky acoustic waves (LSAW), and Bleustein-Gulyaev (BG) waves.

In particular, Love mode sensors consist of a substrate supporting a SH wave mode such as SSBW of ST quartz or the leaky wave of 36° YXLiTaO₃. These modes are converted into a Love-wave mode by application of thin acoustic guiding layer or waveguide. These waves are frequency dependent and can be generated provided that the shear wave velocity of the waveguide layer is lower than that of the piezoelectric substrate. SiO₂ has been used as an acoustic waveguide layer on quartz. Other thermoplastic and crosslinked polymeric waveguide materials such as polymethylmethacrylate, phenol-formaldehyde resin (e.g., trade designation NOVALAC), polyimide and polystyrene, have also been employed.

Alternatively QCM devices can also be used with liquid sample mediums.

Biosensors employing acoustic mechanical means and components of such biosensors are known. See, for example, U.S. Pat. Nos. 5,076,094; 5,117,146; 5,235,235; 5,151,110; 5,763,283; 5,814,525; 5,836,203; 6,232,139. SH-SAW devices can be obtained from various manufacturers such as Sandia National Laboratories, Albuquerque, N. Mex. Certain SH-SAW biosensors are also described in “Low-level detection of a Bacillus anthracis stimulant using Love-wave biosensors of 36° YXLiTaO₃,” Biosensors and Bioelectronics, 19, 849-859 (2004). SAW biosensors, as well as methods of detecting biological agents, are also described in U.S. Patent Application Ser. No. 60/533,169, filed Dec. 30, 2003.

In some embodiments, the surface of the biosensor includes a secondary capture agent or reactant (e.g., antibody) overlying the waveguide layer. In this embodiment, the biosensor typically detects a change in viscosity and/or mass bound by the secondary capture agent or reactant. For this embodiment, the biosensor preferably includes an immobilization layer (overlying the waveguide layer) and optional tie layer(s).

An immobilization layer can be provided for the purpose of binding the secondary capture agent or reactant (e.g., antibody) to the surface. Materials useful for the immobilization layer include those described above.

### Detection Systems and Cartridges

As discussed herein, the materials and methods of the present invention may be used on sensors to provide waveguides, immobilization layers, capture materials, or combinations thereof. The following discussion presents some potential examples of systems and detection cartridges in which the sensors using the materials of the present invention may be used.

FIG. 2 is a schematic diagram of one detection apparatus including a biosensor. The depicted apparatus may optionally include a reagent 322, test specimen 324, wash buffer 326, and liposomes 327. These various components may be introduced into, e.g., a staging chamber 328 where they may intermix and/or react with each other. Alternatively, one or more these components may be present in the staging chamber 328 before one or more of the other components are introduced therein.

For example, it may be desirable that the reagent 322 and the test specimen 324 be introduced into the staging chamber 328 to allow the reagent 322 to act on and/or attach to the target biological analyte within the test specimen 324. Before attachment of the target biological analyte in the test specimen 324 to the liposomes 327, the test specimen 324 may be moved from the staging chamber 328 to the detection chamber 330 where the target biological analyte in the sample material can contact the detection surface 332 of a sensor.

Following the movement of the test specimen 324 from the staging chamber 328 to the detection chamber 330, the liposomes 327 may be introduced into the staging chamber 328, followed by introduction to the detection chamber 330. The liposomes 327 may selectively attach to the target biological analyte material within the detection chamber 330, although they do not necessarily need to do so. The detection surface 332 may preferably be of the type that includes capture agents located thereon such that the target biological analyte and/or the liposomes 327 are selectively attached to the detection surface 332.

In an alternate example, it may be desirable that the reagent 322 and the test specimen 324 be introduced into the staging chamber 328 to allow the reagent 322 to act on and/or attach to the target biological analyte within the test specimen 324. Following interaction of the target analyte with the test specimen 324, the liposomes 327 may be introduced into the staging chamber 328. The liposomes 327 may selectively attach to the target biological analyte material within the staging chamber 328, although they do not necessarily need to do so.

After attachment of the target biological analyte in the test specimen 324 to the liposomes 327, the test specimen 324 (and associated liposomes) may be moved from the staging chamber 328 to the detection chamber 330 where the target biological analyte in the sample material can contact the detection surface 332 of a sensor. The detection surface 332 may preferably be of the type that includes capture agents located thereon such that the target biological analyte in the sample material is selectively attached to the detection surface 332.

It may be preferred that the reagent 322 be selective to the target biological analyte, i.e., that other biological analytes in the test specimen 324 are not modified by the reagent 322. Alternatively, the reagent 322 may be non-selective, i.e., it may act on a number of biological analytes in the test specimen 324, regardless of whether the biological analytes are the target biological analyte or not. In some embodiments, the reagent 322 may preferably be a chemical fractionating agent such as, e.g., one or more enzymes, hypertonic solutions, hypotonic solutions, detergents, etc.

After attachment of the target biological analyte in the test specimen 324 to the liposomes 327, the sample material (with the test specimen 324 and associated liposomes) may be moved from the staging chamber 328 to the detection chamber 330 where the target biological analyte in the sample material can contact the detection surface 332.

In another alternate example, it may be desirable that the liposomes 327 and the test specimen 324 be introduced into the staging chamber 328 to allow the liposomes 327 to attach to the target biological analyte within the test specimen 324. After attachment of the target biological analyte in the test specimen 324 to the liposomes 327, the test specimen 324 (and associated liposomes) may be moved from the staging chamber 328 to the detection chamber 330 where the target biological analyte in the sample material can contact the detection surface 332 of a sensor. The detection surface 332 may preferably be of the type that includes capture agents located thereon such that the target biological analyte and/or liposomes 327 in the sample material is selectively attached to the detection surface 332.

Following movement of the test specimen 324 (and associated liposomes) to the detection chamber 330, reagent 322 may be introduced to the staging chamber 328, and further introduced to the detection chamber 330. In this embodiment, it may be preferred that the reagent 322 be a rupture agent that ruptures the liposomes 327 in detection chamber 330.

Detection of any target biological analytes selectively attached to the detection surface preferably occurs using the sensor 334 as operated by an optional control module 335. The control module 335 may preferably operate the sensor 334 such that the appropriate acousto-mechanical energy is generated. The control module 335 may optionally also set the appropriate flow rate, and also monitor the sensor 334 such that a determination of the presence or absence of the target biological analyte on the detection surface 332 can be made.

Examples of techniques for driving and monitoring acousto-mechanical sensors such as those that may be used in connection with the present invention may be found in, e.g., U.S. Pat. Nos. 5,076,094 (Frye et al.); 5,117,146 (Martin et al.); 5,235,235 (Martin et al.); 5,151,110 (Bein et al.); 5,763,283 (Cernosek et al.); 5,814,525 (Renschler et al.); 5,836,203 ((Martin et al.); and 6,232,139 (Casalnuovo et al.), etc. Further examples may be described in, e.g., Branch et al., “Low-level detection of a Bacillus anthracis simulant using Love-wave biosensors on 36° YX LiTaO₃,” Biosensors and Bioelectronics, 19, 849-859 (2004); as well as in U.S. Patent Application No. 60/533,177, filed on Dec. 30, 2003, and PCT Publication No. WO 2005/066622, titled “Estimating Propagation Velocity Through A Surface Acoustic Wave Sensor”, filed on Dec. 17, 2004.

Although an exemplary detection apparatus that may be used in connection with the present invention is discussed above in connection with FIG. 2, those apparatus may be contained in an integrated unit that may be described as a detection cartridge. Exemplary detection cartridges are further described in PCT Publication No. WO2005/075973 titled “Acousto-mechanical Detection Systems and Methods of Use”, filed Dec. 17, 2004 and PCT Publication No. WO2005/064349, titled “Detection Cartridges, Modules, Systems and Methods”, filed on Dec. 17, 2004, which describe additional features of detection cartridges and/or modules that may be used in connection with the present invention.

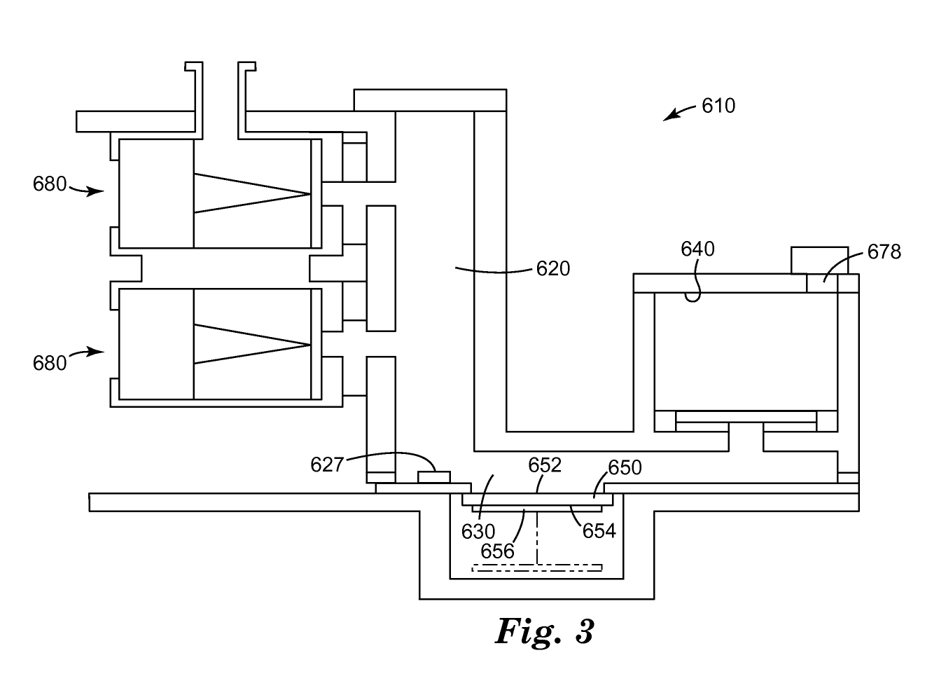

One exemplary embodiment of a detection cartridge 610 including a staging chamber 620, detection chamber 630 and waste chamber 640 is depicted in FIG. 3. The detection cartridge 610 includes a sensor 650 having a detection surface 652 exposed within the detection chamber 630, and an optional magnetic field generator 656, for those applications in which magnetic particles may be used.

It may be preferred that the sensor 650 be an acousto-mechanical sensor such as, e.g., a QCM or a Love mode shear horizontal surface acoustic wave sensor. As depicted, the sensor 650 may preferably be attached such that the backside 654 of the sensor 650 (i.e., the surface facing away from the detection chamber 630) does not contact any other structures within the cartridge 610.

Examples of some potentially suitable methods of attaching acousto-mechanical sensors within a cartridge that may be used in connection with the present invention may be found in, e.g., U.S. Patent Application Ser. No. 60/533,176, filed on Dec. 30, 2003 as well as PCT Publication No. WO 2005/066621, titled “Surface Acoustic Wave Sensor Assemblies”, filed on Dec. 17, 2004.

In some instances, the processes used in the above-identified documents may be used with acoustic sensors that include contact pads that are exposed outside of the boundaries of a waveguide layer on the sensor using a Z-axis adhesive interposed between the sensor contact pads and traces on a carrier or support element to which the sensor is attached. Alternatively, however, the methods described in those documents may be used to make electrical connections through a waveguide layer where the properties (e.g., glass transition point (T_{g}) and melting point) of the Z-axis adhesive and the waveguide material are similar. In such a process, the waveguide material need not be removed from the contact pads on the sensor, with the conductive particles in the Z-axis adhesive making electrical contact through the waveguide material on the contact pads of the sensor.

The embodiment of FIG. 3 includes a vent 678 in the waste chamber 640 that may place the interior volume of the waste chamber 640 in communication with ambient atmosphere. Opening and/or closing the vent 678 may be used to control fluid flow into the waste chamber 640 and, thus, through the cartridge 610. Furthermore, the vent 678 may be used to reduce pressure within the waste chamber 640 by, e.g., drawing a vacuum, etc. through the vent 678.

Although depicted as being in direct fluid communication with the waste chamber 640, one or more vents may be provided and they may be directly connected to any suitable location that leads to the interior volume of the detection cartridge 610, e.g., staging chamber 620, detection chamber 630, etc. The vent 678 may take any suitable form, e.g., one or more voids, tubes, fitting, etc.

Referring again to the cartridge depicted in FIG. 3, the staging chamber 620 may be provided upstream from the detection chamber 630. The staging chamber 620 may provide a volume into which various components may be introduced before entering the detection chamber 630. Although not depicted, it should be understood that the staging chamber 620 could include a variety of features such as, e.g., one or more reagents located therein (e.g., dried down or otherwise contained for selective release at an appropriate time); coatings (e.g., hydrophilic, hydrophobic, etc.); structures/shapes (that may, e.g., reduce/prevent bubble formation, improve/cause mixing, etc.).

Also, the fluid path between the staging chamber 620 and the detection chamber 630 may be open as depicted in FIG. 3. Alternatively, the fluid path between the staging chamber 620 and the detection chamber 630 may include a variety features that may perform one or more functions such as, e.g., filtration (using, e.g., porous membranes, size exclusion structures, beads, etc.), flow control (using, e.g., one or more valves, porous membranes, capillary tubes or channels, flow restrictors, etc.), coatings (e.g., hydrophilic, hydrophobic, etc.), structures/shapes (that may, e.g., reduce/prevent bubble formation and/or transfer, improve mixing, etc.).

Other optional features of the sensor cartridge, such as fluid monitors 627 and modules 680 for delivering various materials are further described in the references described and incorporated by reference herein.

Although the exemplary embodiments described herein may include a single sensor, the detection cartridges of the present invention may include two or more sensors, with the two or more sensors being substantially similar to each other or different. Furthermore, each sensor in a detection cartridge according to the present invention may include two or more channels (e.g., a detection channel and a reference channel). Alternatively, different sensors may be used to provide a detection channel and a reference channel within a detection cartridge. If multiple sensors are provided, they may be located in the same detection chamber or in different detection chambers within a detection cartridge.

Additional discussion related to various detection systems and components (such as detection cartridges including biosensors) may be found in, e.g., U.S. Patent Application No. 60/533,169, filed Dec. 30, 2003; PCT Publication No. WO2005/075973 titled “Acousto-mechanical Detection Systems and Methods of Use”, filed Dec. 17, 2004 and PCT Publication No. WO2005/064349, titled “Detection Cartridges, Modules, Systems and Methods”, filed on Dec. 17, 2004.

### System Design

It may desirable that the detection cartridges of the present invention be capable of docking with or being connected to a unit that may, e.g., provide a variety of functions such as providing power to the sensors or other devices in the detection cartridge, accepting data generated by the sensor, providing the ability to take user input to control fluid flow and/or sensor operation, etc.

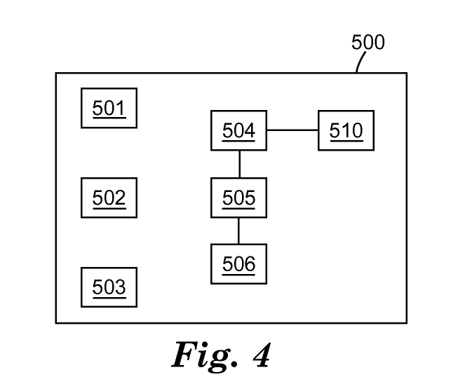

One such system 500 is schematically depicted in FIG. 4, and may preferably include a power source 501 and user interface 502 (e.g., pushbuttons, keyboard, touchscreen, microphone, etc.). The system 500 may also include an identification module 503 adapted to identify a particular detection cartridge 510 using, e.g., barcodes, radio-frequency identification devices, mechanical structures, etc.

The system 500 may also preferably include a sensor analyzer 504 that obtains data from a sensor in the detection cartridge and a processor 505 to interpret the output of the sensor. In other words, sensor analyzer 504 may receive output from a sensor detection cartridge 510 and provide input to processor 505 so that the output of the sensor can be interpreted.

Processor 505 receives input from sensor analyzer 504, which may include, e.g., measurements associated with wave propagation through or over an acousto-mechanical sensor. Processor 505 may then determine whether a target biological analyte is present in sample material. Although the invention is not limited in this respect, the sensor in detection cartridge 510 may be electrically coupled to sensor analyzer 504 via insertion of the detection cartridge 510 into a slot or other docking structure in or on system 500. Processor 505 may be housed in the same unit as sensor analyzer 504 or may be part of a separate unit or separate computer.

Processor 505 may also be coupled to memory 506, which can store one or more different data analysis techniques. Alternatively, any desired data analysis techniques may be designed as, e.g., hardware, within processor 505. In any case, processor 505 executes the data analysis technique to determine whether a detectable amount of a target biological analyte is present on the detection surface of a sensor in detection cartridge 510.

By way of example, processor 505 may be a general-purpose microprocessor that executes software stored in memory 506. In that case, processor 505 may be housed in a specifically designed computer, a general purpose personal computer, workstation, handheld computer, laptop computer, or the like. Alternatively, processor 505 may be an application specific integrated circuit (ASIC) or other specifically designed processor. In any case, processor 505 preferably executes any desired data analysis technique or techniques to determine whether a target biological analyte is present within a test sample.

Memory 506 is one example of a computer readable medium that stores processor executable software instructions that can be applied by processor 505. By way of example, memory 506 may be random access memory (RAM), read-only memory (ROM), non-volatile random access memory (NVRAM), electrically erasable programmable read-only memory (EEPROM), flash memory, or the like. Any data analysis techniques may form part of a larger software program used for analysis of the output of a sensor (e.g., LABVIEW software from National Instruments Corporation, Austin, Tex.).

Still other potentially useful data analysis techniques may be described in the documents identified herein relating to the use of acoustic sensors. Although systems and methods related to the use of surface acoustic wave sensors are described therein, it should be understood that the use of these systems and methods may be used with other acousto-mechanical sensors as well.

### Manufacturing Acousto-Mechanical Sensors

As discussed herein, the present invention relies on the use of acousto-mechanical sensors to detect the presence of target biological analyte within a test sample flowed over a detection surface. Coating or otherwise providing the various materials needed to provide acousto-mechanical sensors with the desired selective attachment properties may be performed using a variety of methods and techniques.

As used with acoustic sensors, the waveguide materials, immobilization materials, capture agents, etc. used on the sensors may be deposited by any suitable technique or method. Typically, it may be preferred that such materials be delivered to a substrate in a carrier liquid, with the carrier liquid and the materials forming, e.g., a solution or dispersion. When so delivered, examples of some suitable deposition techniques for depositing the materials on a surface may include, but are not limited to, flood coating, spin coating, printing, non-contact depositing (e.g., ink jetting, spray jetting, etc.), pattern coating, knife coating, etc. It may be preferred, in some embodiments, that the deposition technique have the capability of pattern coating a surface, i.e., depositing the materials on only selected portions of a surface. U.S. patent application Ser. No. 10/607,698, filed Jun. 27, 2003, describes methods of pattern coating that may be suitable for use in connection with the construction of sensors according to the present invention.

In some embodiments, (such as those described in, e.g., PCT Publication No. WO2005/066092 titled “Acoustic Sensors and Methods”, filed on Dec. 17, 2004 and others), some materials may function as both waveguide material and immobilization material for secondary capture agents on an underlying substrate. In other embodiments, the same materials may function as waveguide material, immobilization material, and capturing material. In both of these variations, the materials of the present invention may preferably be deposited on an underlying substrate that is, itself, effectively insoluble in the carrier liquid such that the carrier liquid does not adversely affect the underlying substrate.

If, however, the surface on which the waveguide materials, immobilization materials, and/or capture agents are to be deposited exhibits some solubility in the carrier liquid used to deliver the material, it may be preferred that the material be deposited using a non-contact deposition technique such as, e.g., ink jetting, spray jetting etc. For example, if the underlying substrate is a waveguide formed of, e.g., polyimide, acrylate, etc., on a sensor substrate and the material of an immobilization layer is to be deposited using, e.g., butyl acetate, as the carrier liquid, then it may be preferred to use a non-contact deposition method to limit deformation of the waveguide and to preferably retain the functional characteristics of the immobilization material exposed on the resulting coated surface. The same considerations may apply to the coating of capture agents on a surface.

There are several variables that may be controlled in a spray-jet coating process, including deposition rate, substrate speed (relative to the spray jet head), sheath gas flow rate, sheath gas, raster spacing, raster pattern, number of passes, percent solids in the sprayed solution/dispersion, nozzle diameter, the carrier liquid, the composition of the underlying surface on which the materials of the present invention are being deposited, etc. Specific conditions under which the materials of the present invention can be deposited to yield a suitable coating may be determined empirically.

The methods of the present invention may be utilized in combination with various materials, methods, systems, apparatus, etc. as described in various U.S. patent applications identified below, all of which are incorporated by reference in their respective entireties. They include U.S. Patent Application Ser. Nos. 60/533,162, filed on Dec. 30, 2003; 60/533,178, filed on Dec. 30, 2003; Ser. No. 10/896,392, filed Jul. 22, 2004; Ser. No. 10/713,174, filed Nov. 14, 2003; Ser. No. 10/987,522, filed Nov. 12, 2004; Ser. No. 10/714,053, filed Nov. 14, 2003; Ser. No. 10/987,075, filed Nov. 12, 2004; 60/533,171, filed Dec. 30, 2003; Ser. No. 10/960,491, filed Oct. 7, 2004; 60/533,177, filed Dec. 30, 2003; 60/533,176, filed Dec. 30, 2003; Ser. No. 11/015,166, titled “Method of Enhancing Signal Detection of Cell-Wall Components of Cells”, filed on Dec. 17, 2004; Ser. No. 11/015,399 titled “Soluble Polymers as Amine Capture Agents and Methods”, filed on Dec. 17, 2004; Ser. No. 11/015,543 titled “Multifunctional Amine Capture Agents”, filed on Dec. 17, 2004; PCT Publication No. WO 2005/066622, titled “Estimating Propagation Velocity Through A Surface Acoustic Wave Sensor”, filed on Dec. 17, 2004; PCT Publication No. WO 2005/066621, titled “Surface Acoustic Wave Sensor Assemblies”, filed on Dec. 17, 2004; PCT Publication No. WO2005/075973 titled “Acousto-mechanical Detection Systems and Methods of Use”, filed Dec. 17, 2004; PCT Publication No. WO2005/064349, titled “Detection Cartridges, Modules, Systems and Methods”, filed on Dec. 17, 2004; and PCT Publication No. WO2005/066092 titled “Acoustic Sensors and Methods”, filed on Dec. 17, 2004.

### EXAMPLES

These examples are merely for illustrative purposes only and are not meant to be limiting on the scope of the appended claims. All parts, percentages, ratios, etc. in the examples and the rest of the specification are by weight, unless noted otherwise.

### Example 1

### Preparation of Surface Acoustic Wave Sensors

Delay line shear-horizontal surface acoustic wave (SH-SAW) sensors could be obtained from Com Dev (Cambridge, Ontario, Canada). The sensors could be coated with a 50:50 (methyl methacrylate/isobornyl methacrylate) copolymer waveguide, such as the one described in Example W1 of PCT Publication No. WO2005/066092 titled “Acoustic Sensors and Methods”, filed on Dec. 17, 2004. The waveguide-coated sensors could be subsequently coated with a terpolymer immobilization chemistry consisting of isobornyl methacrylate, methyl methacrylate and hydroxyethyl methacrylate glutaroylsaccharin, such as the one described in Example MP26 of PCT Publication No. WO2005/066092 titled “Acoustic Sensors and Methods”, filed on Dec. 17, 2004.

Biotin-amine could be immobilized onto the active channel of the sensor using chemistries and hand-coating or sprayjet-coating processes known in the art. A non-specific Chicken IgY could be obtained from, for example, Jackson ImmunoResearch Laboratories Inc. (West Grove, Pa.) and hand-coated or sprayjet-coated onto the reference channel of the sensor.

The coated sensors could be heat-bonded to flexible circuits via a conductive adhesive. The bonded sensors could be attached to a temperature-controlled flowpod via a double-sided adhesive film. The assembled sensors could be connected to an electronic measurement board driven by a software program using a network analyzer. The software could be used to collect signal attenuation and phase data in the desired frequency range throughout the experiments.

### Example 2

### Surface Acoustic Wave Experimental Parameters and Data Collection

A syringe pump could be used to flow Phosphate-buffered Saline (PBS), pH 7.4, buffer over the sensor at a desired flow rate. After sufficient stabilization of the buffer flow, the sample could be injected into the device and allowed to flow over the sensor surface. The operating frequency of the sensor devices could be 103 MHz. Phase and attenuation signals could be collected until the experiment is complete.

A time gating algorithm, such as the one described in the 8753ET/ES Network Analyzers User's Guide (Agilent Technologies, pp 3-35 to 3-36), could be used to process the raw phase and attenuation data. The time interval unit for data collection could be set between 8-15 seconds. The raw data could be collected and time gating could be done using a software program written in, for example, Matlab (The Mathworks, Natick, Mass.). The time gated data could be analyzed to calculate shifts in phase and attenuation. All of the data processing could be done using Matlab software.

### Example 3

### Liposome Preparation

Approximately 50 mL of a 20 mg/mL solution of DPPC (1,2-dipalmitoyl-sn-glycero-3-phosphocholine, Avanti Polar Lipids, Alabaster, Ala.) in chloroform could be reduced to dryness and could be subsequently mixed with 2 mL of a 50 mg/mL solution of 16:0 Biotinyl-Cap-PE (1,2-dipalmitoyl-sn-glycero-3-phosphoethanolamine-N-(Cap-biotinyl), sodium salt, Avanti Polar Lipids, Alabaster, Ala.) in chloroform. The resulting mixture could be made up to 10 mL with chloroform. This solution would contain approximately 110 mg/mL lipid mixture (˜11% biotinylated). A portion (4.4 mL) of this lipid mixture could be reduced to dryness on a rotary evaporator. The solid residue could be hydrated with 20 mL of a 0.1M solution of dibenzoylcystine (DBC, sodium salt) in water, and then sonicated in a Branson 3510 ultrasonic water bath for 1 hour at 45° C. This mixture could be left standing for three days at room temperature.

The aforementioned solution (20 mL) could be mixed with a 0.5 mL of a prefiltered solution of Chrome Azurol S (CAS—13 mg/mL in phosphate buffered saline (PBS)) and sonicated at 50° C. for 15 minutes with the equipment described above. Four 1.0 mL aliquots each could be extruded 15 times through 19 mm diameter polycarbonate track-etch membranes using an Avanti Mini-Extruder (Avanti Polar Lipids). Two aliquots could be extruded, for example, through 100 nm membranes, and two could be extruded through 800 nm membranes. Each aliquot then could be passed through a HiTrap desalting column (GE Healthcare, Uppsala, Sweden), using PBS as eluant, to remove unencapsulated dibenzoylcystine.

A 0.5% (w/v) succinic acid solution could be prepared in PBS. The liposome lysing solution could be prepared by addition of 25% (v/v) TRITON-X 100, typically in a ratio of 7 μL of a 25% w/w solution (in water) for every mL of liposome solution. In the presence of succinic acid, the DBC anion that could be released from the liposomes anion would be protonated to form neutral DBC, which gels water at concentrations above 4 mmol.

### Example 4

### Detection of Streptavidin Using a SH-SAW Sensor

This test could confirm the sensor response to 1 μg/ml of streptavidin and establish a baseline sensor response. Streptavidin could be obtained from (Jackson ImmunoResearch, West Grove, Pa.) and could be dissolved in PBS to a final concentration of 1 microgram/mL. The SAW sensor would be prepared as described in Example 1. Liposomes would be prepared as in Example 2. The sample flow rate could be set to 30 μl/min. It would take approximately 150 time points for each injected sample to pass over the sensor. The phase and attenuation response would be calculated as the difference between the active channel and reference channel.

A streptavidin sample could be injected at time point 200 and the sensor response would be observed as a decrease in phase following the injection. The data would show the phase shift due to the binding of the 1 μg/ml streptavidin to the biotin coated on the sensor surface.

### Example 5

### Streptavidin Detection with Biotinylated Liposomes in a SH-SAW Sensor

Streptavidin could be dissolved in PBS to a final concentration of 1 microgram/mL. The SAW sensor would be prepared as described in Example 1. Liposomes would be prepared as in Example 2. The sample flow rate could be set to 30 μl/min. After each injected sample is allowed to pass over the sensor, the phase and attenuation response could be calculated as the difference between the active channel and reference channel.

After stabilization of the sensor signal, a 500 μL aliquot of streptavidin (1 μg/mL) could be injected into the sensor (approximately, at time point 150). The sensor could be allowed to equilibrate until time point about 450, at which time the liposome solution could be injected. Significant responses in phase shift and attenuation would be observed, due to the liposomes binding to the sensor. The phase shift would remain as the liposome solution is replaced with the run buffer, indicating that at least some of the liposomes and/or their contents remain associated with the sensor surface. At a time point of approximately 750, the succinic acid/TRITON-X 100 solution could be injected into the sensor. This solution, which is known to lyse the liposomes and cause gelling of DBC, would result in significant responses in both the phase and attenuation of the sensor signal.

### Example 6

### Preparation of Biotinylated Liposome

A chloroform solution of DPPC (1.0 mL at 25 mg/mL) (1,2-dipalmitoyl-sn-glycero-3-phosphocholine available from Avanti Polar Lipids, Alabaster, Ala.) was added to DOPC (1,2-dioleoyl-sn-glycero-3-phosphocholine available from Avanti Polar Lipids, Alabaster, Ala.) (5 mg) in a small round bottomed flask. N-biotinyl-Cap-PE (1,2-dioleoyl-sn-glycero-3-phosphoethanolamine-N-(Cap-Biotinyl) (sodium salt) available from Avanti Polar Lipids, Alabaster, Ala.) as 5 μL of a 50 mg/mL CHCl₃ solution was added and then the solvent was removed on a rotary evaporator with gentle heating, followed by evacuation on a vacuum line at ˜0.2 torr and room temperature.

The dried lipid film was then hydrated with 1 mL of a solution of DBC (dibenzoylcystine) in water (100 mmol), and heated to 50° C. in an ultrasonic bath for 60 minutes. The DBC was prepared from cystine and benzoyl chloride under phase transfer conditions (ref Menger, Fredric M.; Caran, Kevin L. J. Amer. Chem. Soc. (2000), 122(47), 11679-11691) before addition to the dried lipid film in solution form.

The flask was then subjected to 5 freeze-thaw cycles by alternately dipping it in a dry ice/acetone bath until frozen and then thawing in a warm water bath. The liposome solution was then passed fifteen times through an Avanti Mini-Extruder (Avanti Polar Lipids) equipped with a polycarbonate membrane with 100 nm pores. Finally, the sample was passed through a HiTrap desalting column (GE Healthcare, Piscataway, N.Y.) and the cloudy fractions retained. Lipid concentration (measured by Phospholipid C test, Wako Chemicals, Richmond, Va.) was 14.2 mg/mL, and z-average particle size was 107 nm (measured by Malvern Zetasizer Nano, Malvern Instruments, Worcestershire, UK).

### Example 7

### Preparation of Liposome without Biotinylation

A chloroform solution of DPPC (1.0 mL at 25 mg/mL) was added to DOPC (5 mg) in a small round bottomed flask. The solvent was removed on a rotary evaporator with gentle heating, followed by evacuation on a vacuum line at ˜0.2 torr and room temperature.

The dried lipid film was then hydrated with 1 mL of a solution of DBC in water (100 mmol), and heated to 50° C. in an ultrasonic bath for 60 minutes. The flask was then subjected to 5 freeze-thaw cycles by alternately dipping it in a dry ice/acetone bath until frozen and then thawing in a warm water bath. The liposome solution was then passed eleven times through an Avanti Mini-Extruder (Avanti Polar Lipids) equipped with a polycarbonate membrane with 100 nm pores. Finally, the sample was passed through a HiTrap desalting column (GE Healthcare, Piscataway, N.Y.) and the cloudy fractions retained. Lipid concentration (measured by Phospholipid C test, Wako Chemicals, Richmond, Va.) was 18.1 mg/mL, and z-average particle size was 106 nm (measured by Malvern Zetasizer Nano, Malvern Instruments, Worcestershire, UK).

### Example 8

### Streptavidin Detection with Biotinylated Liposomes in a QCM Sensor

Four gold-coated QCM crystals (Q-Sense, Glen Burnie, Md.) were cleaned with H₂O₂/NH₃/H₂O 1:1:5 at 80° C. for ˜10 min., then irradiated with UV (UV TipCleaner, Bioforce Nanosciences, Ames Iowa) and spin-coated (10 sec., 2000 rpm) with an ethanol solution of biotinylated thiol (HS-C₁₁NH(O)-Biotin (available from Prochimia Surfaces, Sopot, Poland), dissolved at 1% w/w in ethanol). The coated QCM crystals were then placed in a QD-4 quartz crystal microbalance (Q-Sense, Glen Burnie, Md.).

Air was passed over the four crystals for 10 minutes, followed by a buffer solution passed over the crystals at 10 μL/min. The buffer solution was 10 mmolar N-[tris(hydroxymethyl)methyl]-2-aminoethanesulfonic acid (commercially available from Sigma Chemical Co., St Louis, Mo.), adjusted to pH 6.5 with 0.1 N HCl and NaOH (hereinafter referred to as “TES”).

The same flow rate of 10 μL/min was used for all subsequent solutions. After an equilibration time (˜10.5 min), the inlet tubes were transferred to a solution of 2.1 mg streptavidin (available from Jackson ImmunoResearch, (West Grove, Pa.)) in 10 mL TES.

After 9 minutes, the inlet tubes were returned to 10 mmol TES for 6.5 minutes to rinse the crystal cell, and then exposed to TES solutions of liposomes from example 6 (in crystals 1 and 4) and Example 7 (in crystals 2 and 3). All liposome solutions were diluted to 1.2 mg/mL lipid in 10 mmol TES prior to introduction to the crystals.

After a further 15 minutes, the inlet tubes were returned to 10 mmol TES for 15 minutes and then to a solution of citric acid (Bio-Rad, Hercules, Calif.) dissolved at 1% w/w in DI water (crystals 1 and 2) or 0.25% TRITON X-100 ((Sigma Chemical Co., St Louis, Mo.) dissolved at 1% w/w in DI water) in 1% citric acid (crystals 3 and 4). After a further 23 minutes, the crystals were returned to TES, then DI water, and finally allowed to run dry.

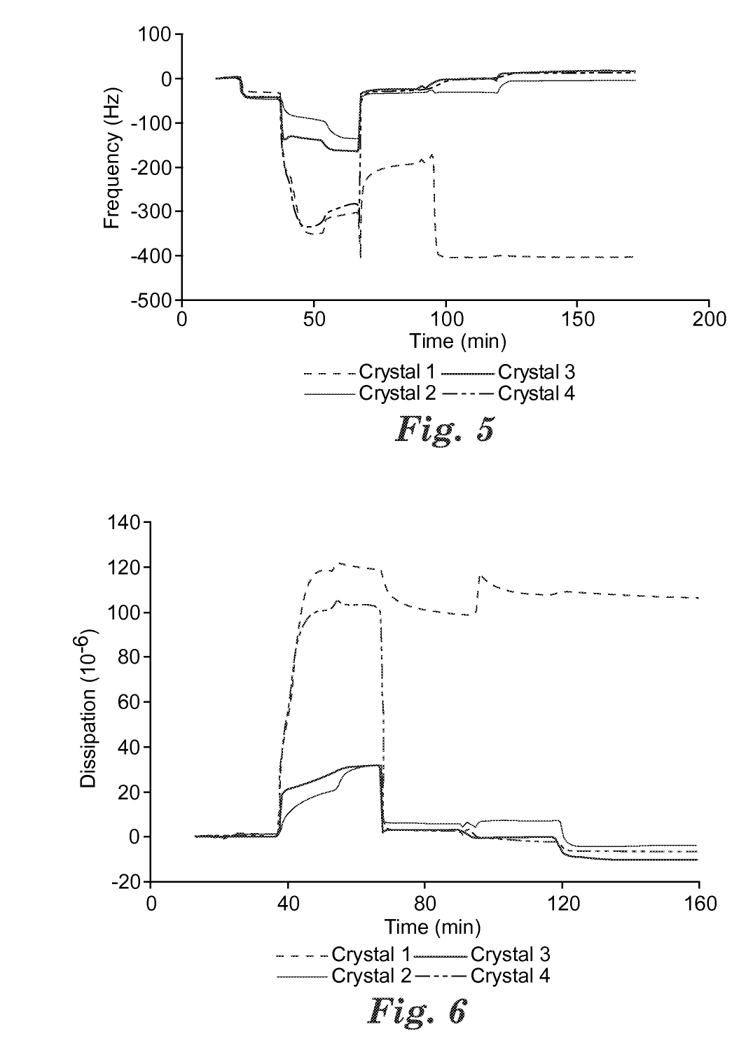

The frequency and dissipation data of the four crystals are depicted graphically in FIGS. 5 and 6. Although data collection began in air, the data was re-normalized for presentation in FIGS. 5 and 6 to make the zero point in buffer.

FIG. 5 displays frequency changes for the third harmonic frequency of the four crystals throughout the course of Example 8. Addition of streptavidin caused a distinct change in frequency (40-50 Hz) (crystals 1-4). Addition of the streptavidin-binding biotinylated liposomes increased the magnitude of the frequency shift (330-350 Hz) substantially (crystals 1 and 4). The frequency shift with the biotinylated liposomes dissipated when the crystal was treated with the TRITON-X 100 solution (crystal 4). However, the large frequency shift caused by the streptavidin-binding biotinylated liposomes was retained with addition of the citric acid solution and eventually increased to 400 Hz after replacement of the citric acid solution with TES (crystal 1).

Non-biotinylated liposomes also led to increased frequency shift (up to 160 Hz), however this increased frequency effect dissipated after addition of the citric acid solution, with or without TRITON-X 100 (crystals 2 and 3).

FIG. 6 displays analogous changes in dissipation. Addition of streptavidin caused only a slight change in dissipation for all four crystals (<2×10⁻⁶). Addition of non-biotinylated liposomes caused a more-than 10 fold increase in dissipation (˜32×10⁻⁶) (crystals 2 and 3), and biotinylated liposomes up to 120×10⁻⁶ (crystals 1 and 4). While not intending to be bound by theory, the effects may be attributed to the citric acid solution removing the non-specifically bound liposomes that while addition of TRITON-X 100 potentially lysed the streptavidin-binding liposomes and returned the dissipation values to near their original levels. The biotinylated liposomes that were not exposed to TRITON-X 100 eventually settled at about 106×10⁻⁶ (crystal 1).

All references and publications identified herein are expressly incorporated herein by reference in their entirety into this disclosure. Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A method of detecting a target biological analyte, the method comprising:
providing a system comprising an acousto-mechanical device comprising a detection surface with a capture agent located on the detection surface, wherein the capture agent is capable of selectively attaching the target biological analyte to the detection surface;
contacting the detection surface of the acousto-mechanical device with a sample material that may contain the target biological analyte;
selectively attaching the target biological analyte to the detection surface;
contacting the target biological analyte and/or detection surface with a liposome; and
operating the acousto-mechanical device to detect the attached target biological analyte while the detection surface is submersed in liquid.

2. A method according to claim 1, wherein the liposome modifies the rheological properties of the fluid near the sensor surface.

3. A method according to claim 1, wherein the liposome changes the mass attached to the surface.

4. A method according to claim 1, wherein the liposome modifies the dielectric properties of the fluid near the sensor surface, the sensor surface itself and/or any intervening layers on the sensor surface.

5. A method according to claim 1, wherein the liposome comprises 1,2-dipalmitoyl-sn-glycero-3-phosphocholine (DPPC), 1,2-dipalmitoyl-sn-glycero-3-phosphoethanolamine-N-(Cap-biotinyl) (16:0 Biotinyl-Cap-PE), and combinations thereof.

6. A method according to claim 1, wherein the liposome ruptures upon contact with the target biological analyte and/or the detection surface.

7. A method according to claim 1, further comprising the step of contacting the liposome with a rupture agent.

8. A method according to claim 1, wherein the acousto-mechanical device comprises a surface acoustic wave device.

9. A method according to claim 8, wherein the surface acoustic wave device comprises a shear horizontal surface acoustic wave device.

10. The method of claim 1, further comprising the step of fractionating target biological analyte located within the sample material.

11. A method according to claim 10, wherein the fractionating comprises chemically fractionating the target biological analyte in the sample material.

12. A method according to claim 10, wherein the fractionating comprises mechanically fractionating the target biological analyte in the sample material.

13. A method according to claim 10, wherein the fractionating comprises thermally fractionating the target biological analyte in the sample material.

14. A method according to claim 10, wherein the fractionating comprises electrically fractionating the target biological analyte in the sample material.

15. The method of claim 1, wherein the step of contacting the target biological analyte with a liposome comprises contacting the sample material with liposomes, wherein a target biological analyte within the sample material interacts with the liposomes such that the target biological analyte is bound to the liposomes within the sample material prior to contacting the detection surface.

16. The method of claim 1, wherein the acousto-mechanical device is a quartz crystal microbalance device.
